(19) United States
(12) Patent Application Publication
(10) Pub. No.: US 2011/0004294 A1
(43) Pub. Date: Jan. 6, 2011

(54) FATIGUE-RESISTANT STENT

(75) Inventor: Michael R. Bialas, Temecula, CA (US)

Correspondence Address: WORKMAN NYDEGGER, 1000 EAGLE GATE TOWER,, 60 EAST SOUTH TEMPLE, SALT LAKE CITY, UT, 84111, US

(73) Assignee: ABBOTT LABORATORIES, Abbott Park, IL (US)

(21) Appl. No.: 12/497,084

(22) Filed: Jul. 2, 2009

(51) Int. Cl. A61F 2/06

(52) U.S. Cl. 623/1.17; 623/1.23; 623/1.18

(57) ABSTRACT

A fatigue-resistant implantable medical device. The fatigue-resistant implantable medical device includes a tubular stent. The tubular stent can have a laser-cut length and a shape-set length that is either longer or shorter than the laser-cut length. Such stents will tend to return to their laser-cut length when they are compressed for delivery. A stent that is shape-set longer than its laser-cut/delivery length that is deployed in a vessel that is shortened for stent delivery will tend to return to its shape-set length when the vessel returns to its neutral (i.e., elongated) length. Likewise, a stent that is shape-set shorter than its laser-cut/delivery length that is deployed in a vessel that is elongated for stent delivery will tend to return to its shape-set length when the vessel returns to its neutral (i.e., shortened) length. Such stents will experience reduced axial loading and reduced mean strain, thereby improving fatigue life.

### BACKGROUND OF THE INVENTION

1. The Field of the Invention

The present invention relates to fatigue resistant medical devices. More particularly, the invention relates to a fatigue resistant stent having reduced mean strain when installed in a body lumen.

2. The Relevant Technology

Stents, grafts, endoprostheses and a variety of other implantable medical devices are well known and used in interventional procedures, such as for treating aneurysms, for lining or repairing vessel walls, for filtering or controlling fluid flow, and for expanding or scaffolding occluded or collapsed vessels. Such implantable medical devices can be delivered and used in virtually any accessible body lumen of a human or animal, and can be deployed by any of a variety of recognized means.

One recognized indication of an implantable medical device, such as a stent, is for the treatment of atherosclerotic stenosis in blood vessels. For example, after a patient undergoes a percutaneous transluminal coronary angioplasty or similar interventional procedure; a stent is often deployed at the treatment site to improve the results of the medical procedure and reduce the likelihood of restenosis. The stent is configured to scaffold or support the treated blood vessel; if desired, it can also be loaded with a beneficial agent so as to act as a delivery platform to reduce restenosis or the like.

Other suitable examples of medical conditions for which implantable medical devices are an appropriate treatment include, but are not limited to, arterial aneurysms, venous aneurysms, coronary artery disease, peripheral artery disease, peripheral venous disease, chronic limb ischemia, blockage or occlusion of the bile duct, esophageal disease or blockage, defects or disease of the colon, tracheal disease or defect, blockage of the large bronchi, blockage or occlusion of the ureter, or blockage or occlusion of the urethra.

An implantable medical device, such as a stent, is typically delivered by a catheter delivery system to a desired location or deployment site inside a body lumen of a vessel or other tubular organ. The intended deployment site may be difficult to access by a physician and often involves traversing the delivery system through a tortuous luminal pathway. Thus, it can be desirable to provide the implantable medical device with a sufficient degree of flexibility during delivery to allow advancement through the anatomy to the deployment site. Moreover, it may be desirable for the implantable medical device to retain structural integrity while flexing and bending during delivery so that cracks do not form.

Current stent designs are typically composed of a series of repeated rings that are connected in series. Once deployed, the stent (such as in a Superficial Femoral Artery (“SFA”) application) undergoes longitudinal, bending, torsional, tensional, and radial cyclical loading that can lead to fatigue failures. The stent connection sections or connection elements that join the rings also transmit stress from ring to ring when the SFA lengthens and shortens during normal leg movement. In addition, when the stent goes around a curve the connecting elements or sections require the portions of the ring apposed to the outside of the curve to lengthen and the portions of the ring apposed to the inside of the curve to shorten. Lengthening and shortening portions of the ring increase the maximum stress because the ring cannot expand evenly promoting fatigue failures.

Current designs of implantable medical devices that are subjected to these types of forces often fail. Failure can result in crack formation and possible stent fracture. In the event of stent fracture, the sharp edges may puncture the vessel, muscle tissue or cause bleeding. Consequently, the fractured stent may cause thrombus formation or blockage within the vessel or other problems.

### BRIEF SUMMARY OF THE INVENTION

Embodiments of fatigue-resistant implantable medical devices, methods for their manufacture, and methods for their deployment are disclosed.

A fatigue-resistant implantable medical device (e.g., a stent) can be laser-cut to form a series of patterned rings connected by links. The stent can then expanded and shape-set at a length that is either shorter or longer than its laser-cut length, depending on the application. The material from which the stent is made will have essentially zero strain in its unconstrained, shape-set state. As the stent is collapsed for deployment, however, the laser-cut pattern causes the stent to return to its laser-cut length. When the stent is deployed in a vessel, the rings of the stent expand radially and engage with the walls of the vessel or other lumen. Because the rings of the stent engage with the walls of the vessel, the deployed stent is deployed at a length essentially equal to its collapsed length but different than its shape-set (i.e., zero strain) length.

Because many vessels, such as the superficial femoral artery (“SFA”), are elongated for stent deployment, a stent that is deployed at a length longer than its shape-set length will typically be able to return to its shape-set length when the vessel returns to its natural length. In another application, it may be desirable to compress or shorten a vessel prior to stent deployment. In such an application, a stent that is deployed at a length shorter than its shape-set length will typically be able to return to its shape-set length when the vessel returns to its natural length. Such stents will experience less or no mean strain as the vessels elongates and shortens during normal body movement, thereby increasing the fatigue life of the stents.

In one embodiment, a fatigue-resistant implantable medical device is disclosed. The fatigue-resistant implantable medical device includes a tubular stent that includes a multiplicity of separate cuts that permit the tubular stent to expand to provide scaffolding support to a lumen. The tubular stent can have an unconstrained configuration that includes a first length and a constrained configuration that includes a second length that is greater than or less than the first length.

In one embodiment, the unconstrained configuration can include a shape-set and expanded state. That is, because of the characteristics of shape-memory materials, shape-setting the expanded stent produces a state where the stent has no appreciable tendency to expand or contract. As a result, when the tubular stent is radially compressed onto a delivery catheter by a delivery sheath stent, it is said to be constrained. Thus, in one embodiment, the constrained configuration can include a delivery configuration. Likewise, because a deployed stent is constrained to some degree by the lumen in which it is deployed, the constrained configuration can include a deployed configuration.

In one embodiment, the second length (i.e., the length in the constrained configuration) can be about 2% to about 15% different (i.e., longer or shorter) than the first length (i.e., the length in the unconstrained configuration). In another embodiment, the second length can be about 3% to about 10% different (i.e., longer or shorter) than the first length or, in another embodiment, the second length can be about 5% different (i.e., longer or shorter) than the first length.

In one embodiment, the tubular stent is formed from a shape-memory and/or a super-elastic material. In one embodiment, the shape-memory and/or a super-elastic material can include a nickel-titanium alloy. In one embodiment, the tubular stent can be a self-expanding stent.

In one embodiment, a method of manufacturing a fatigue-resistant implantable medical device can include (1) providing a tubular stent configured to expand between a first configuration and a second configuration, the first configuration including a first diameter and a first length, (2) expanding the tubular stent to the second configuration, the second configuration including a second diameter that is larger than the first diameter and a second length that is shorter or longer than the first length, (3) heat setting the tubular stent in the second configuration, and (4) radially compressing the tubular stent to return the tubular stent to the first configuration.

In one embodiment, the radially compressed stent can further include a delivery configuration wherein the tubular stent is radially compressed onto a delivery catheter by a delivery sheath.

In one embodiment, a method of deploying a fatigue-resistant implantable medical device is disclosed. The method can include (1) providing a tubular stent configured to transition between a lengthened configuration and a shortened configuration, the tubular stent being shape-set in an elongated or a shortened configuration such that the shape-set configuration has substantially no lengthwise strain (2) providing a body lumen that can transition between an elongated state, a shortened state, and a mean state that is between the elongated state and the shortened state, and (3) deploying the tubular stent in the elongated or the shortened configuration in a body lumen with the body lumen in the elongated state or the shortened state such that the deployed stent has substantially no lengthwise strain when the body lumen is in the mean state.

These and other advantages and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates a planar side view of a portion of a fatigue-resistant implantable medical device according to one embodiment of the present invention;

FIG. 2A illustrates a side view of a fatigue-resistant implantable medical device in a compressed configuration;

FIG. 2B illustrates a side view of the fatigue-resistant implantable medical device shown in FIG. 2A in an expanded configuration;

FIG. 2C illustrates a perspective view of the fatigue-resistant implantable medical device shown in FIG. 2B;

FIG. 3A illustrates a fatigue-resistant implantable medical device having a first length;

FIG. 3B illustrates the fatigue-resistant implantable medical device of FIG. 3A in an expanded and shape-set configuration having a second length that is shorter than the first length;

FIG. 3C illustrates the fatigue-resistant implantable medical device of FIG. 3B in a compressed and elongated delivery configuration with a length that is substantially equal to the first length;

FIG. 3D illustrates the fatigue-resistant implantable medical device of FIG. 3C in a partially deployed configuration;

FIG. 3E illustrates the fatigue-resistant implantable medical device of FIG. 3D deployed in a vessel in an elongated configuration with a length that is substantially equal to the first length; and

FIG. 3F illustrates the fatigue-resistant implantable medical device of FIG. 3E deployed in a vessel with the vessel in a relaxed state with implantable medical device having a length that is substantially equal to the second (i.e., unconstrained, shape-set) length.

### DETAILED DESCRIPTION OF THE PREFERRED EMBODIMENTS

### I. Introduction

Embodiments of fatigue-resistant implantable medical devices, methods for their manufacture, and methods for their deployment are disclosed. Fatigue-resistant implantable medical devices such as stents are often manufactured by laser-cutting the stent pattern out of small diameter tubes. Stents can also be formed from sinusoidal rings that are connected by links. Embodiments of the invention reduce the average strain imposed on stents by axial compression.

Implantable medical devices that are deployed in the body can be subjected to forces that can cause the devices to fatigue and fail. In some instances, implantable medical devices are subject to compression including axial compression. For example, implantable medical devices such as stents are often used in arteries and other body lumens to open blocked regions and/or to provide scaffolding to the lumen.

For instance, the superficial femoral artery (“SFA”) can become occluded due to peripheral arterial disease and is commonly treated with a combination of balloon angioplasty and nitinol self-expanding stents to return normal blood flow to the lower extremities. Since this treatment began, it has become apparent that the SFA undergoes significant deformation during leg movement that places significant loads on stents implanted in this vessel. These mechanically challenging conditions have led to the fracture of a significant percentage of stents implanted in the SFA. Although the exact clinical significance of these fractures can vary from patient to patient, these fractures are undesirable because they can, for example, lead to thrombus or vessel dissection and it is in the patient's best interest to implant a stent that will resist this sort of failure.

One of the most challenging deformations or loads experienced by stents is axial compression. In an SFA procedure, for example, stents are typically deployed in the SFA while the patient's leg is fully extended. Extending the leg typically facilitates stent deployment by, for example, elongating the vessel and reducing the tortuousness of the path that the stent and the stent delivery catheter have to traverse in order to reach the deployment site. However, deploying the stent in an elongated vessel can have clinical implications.

For instance, as the patient's leg moves during normal leg movement, the SFA elongates and shortens applying loads that cause the stent to elongate and shorten as well. In extreme cases, stents implanted in the SFA are thought to lengthen and/or shorten axially by as much as 10% during normal leg movement. This repeated motion can lead to fatigue fractures in the stents including the stent's struts.

Embodiments of the present invention are intended to reduce the strain imposed on the stent by axial deformation by providing for the deployment of stent in an elongated or shortened state (depending on whether the vessel is elongated or shortened during deployment) such that stent returns to its natural, unconstrained length when the vessel returns to its natural length. Such a stent will experience reduced axial loading and a reduced overall percentage of length change as the deployment vessel naturally elongates and shortens. Reducing the mean strain imposed on a stent by compression and elongation of a patient's anatomy can, for example, improve the fatigue life of the stent. A stent having an improved fatigue life will tend to have a longer functional lifespan and such a stent is less likely to fail through crack formation and/or fracture.

Moreover, reducing the overall length change (e.g., 105% to 95% versus 100% to 90%) experienced by a stent as the deployment vessel naturally elongates and shortens can widen the design window for stent patterns. The basic premise being that the more length change that a stent has to tolerate, the more difficult it is to generate a design that meets this parameter in addition to all the other deign criteria, such as stent pattern, vessel coverage, crush resistance, radial outward force, strut width, strength vs. ductility of the stent material, and/or the expansive force of the stent against the vessel wall While both stents in the above presented example are cycling over a 10% length change, the stent that is cycling between 105% and 95% of its unconstrained length is only experiencing a length change of 5% in one direction or the other, whereas the stent that is cycling between 100% and 90% of its unconstrained length is experiencing a length change of −10% in one direction. Because the maximum directional length change is shifted to 5% from 10%, a stent designer can design a stent to tolerate a 5% length change as opposed to having to accommodate a 10% length change.

In one embodiment, a fatigue-resistant implantable medical device (e.g., a stent) can be laser-cut to form a series of patterned rings connected by links. The stent can then be expanded and shape-set at a length that is longer or shorter than its laser-cut length depending on whether the vessel where the stent is to be deployed is elongated or shortened for deployment. The material from which the stent is made will have essentially zero strain in its unconstrained, shape-set state. As the stent is collapsed for deployment, however, the laser-cut pattern can force the stent to return to its laser-cut length when it is compressed onto a delivery catheter. When the stent is deployed in a vessel, the rings expand radially and engage with the walls of the vessel fixing the deployed stent at a length essentially equal to its collapsed length but different (i.e., longer or shorter) than its shape-set (i.e., zero strain) length. However, because vessels such as the SFA are typically elongated for stent deployment, a stent that is deployed at a length longer than its shape set length will typically be able to return to its shape-set length when the vessel returns to its natural length. Such a stent will experience little or no mean strain as the vessel or lumen elongates and shortens during normal body movement, thereby increasing the fatigue life of the stent. The opposite is also true where the vessel is shortened for stent deployment. However, in such a case, it would be desirable to shape-set the stent at a length longer than its laser-cut or deployed length so that the stent returns to its unconstrained length when the vessel lengthens to return to it natural length.

### II. Fatigue-Resistant Implantable Medical Devices

In accordance with the present invention, a fatigue-resistant implantable medical device can be provided for delivery within a body lumen of a human or other animal. Examples of fatigue-resistant implantable medical devices can include stents, filters, grafts, valves, occlusive devices, trocars, aneurysm treatment devices, PFO closure devices, or the like. Additionally, a fatigue-resistant implantable medical device can be configured for a variety of intralumenal applications, including vascular, coronary, biliary, esophageal, urological, gastrointestinal, or the like. In instances where a deployment site is elongated or shortened prior to medical device deployment, fatigue-resistant implantable medical devices can be prepared such that their shape-set length is slightly different (i.e., less than or greater than) than their deployed dimensions. As such, fatigue-resistant implantable medical devices according to the present disclosure can have reduced strain when the patient's anatomy returns to its normal (i.e., not elongated or shortened) state and, as such, they can better withstand stresses or strains produced by repeated elongation and shortening of the deployment site as the body moves.

In one embodiment, a fatigue-resistant implantable medical device can include a stent having at least a first set of interconnected strut elements that cooperatively define an annular element or sub-endoprosthesis. A strut element can be more generally described as an endoprosthetic element, wherein all well-known endoprosthetic elements can be referred to here as a “strut element” for simplicity. Usually, each strut element can be defined by a cross-sectional profile as having a width and a thickness, and including a first end and a second end bounding a length. The stent element can be substantially linear, arced, rounded, squared, combinations thereof, or other configurations. The strut element can include a bumper, crossbar, connector, interconnector, intersection, elbow, foot, ankle, toe, heel, medial segment, lateral segment, coupling, sleeve, combinations thereof, or the like, as described in more detail below. The strut element can have improved structural integrity by including crack-inhibiting features, which are described in detail in the incorporated references.

Usually, the annular elements or sub-endoprosthesis can include a plurality of circumferentially-adjacent crossbars that are interconnected end-to-end by an elbow connection, intersection, or a foot extension. As such, at least one annular element or sub-endoprosthesis can include an elbow, intersection, or a foot extension (“foot”) extending between at least one pair of circumferentially-adjacent crossbars. The elbow or foot can thus define an apex between the pair of circumferentially-adjacent crossbars of the annular element or sub-endoprosthesis. Also, an intersection can have a shape similar to a crossbar or interlinked crossbars so as to provide a junction between two coupled pairs of circumferentially-adjacent crossbars.

The elbow can be configured in any shape that connects adjacent ends of circumferentially-adjacent crossbars, and can be described as having a U-shape, V-shape, W-shape, L-shape, X-shape, Y-shape, H-shape, K-shape, or the like. The elbow and/or intersection can be configured in any shape that connects longitudinal and circumferentially adjacent crossbars, and can be described as having a cross shape, X-shape, Y-shape, W-shape, H-shape, K-shape, or the like. The foot can have a foot shape having a first foot portion extending circumferentially from an end of one of the adjacent strut members and a second foot portion extending circumferentially from a corresponding end of the other of the circumferentially-adjacent strut members. In combination, the first and second foot portions generally define an ankle portion connected to a toe portion through a medial segment and the toe portion connected to a heel portion through a lateral segment.

As described herein, a fatigue-resistant implantable medical device, in one configuration, can include two or more interconnected annular elements or sub-endoprosthesis. Each annular element or sub-endoprosthesis can generally define a ring-like structure extending circumferentially about a longitudinal or central axis. The cross-sectional profile of each annular element or sub-endoprosthesis can be at least arcuate, circular, helical, or spiral, although alternative cross-sectional profiles, such as oval, oblong, rectilinear or the like, can be used. The different annular elements can be defined as having the same characterization or different characterizations.

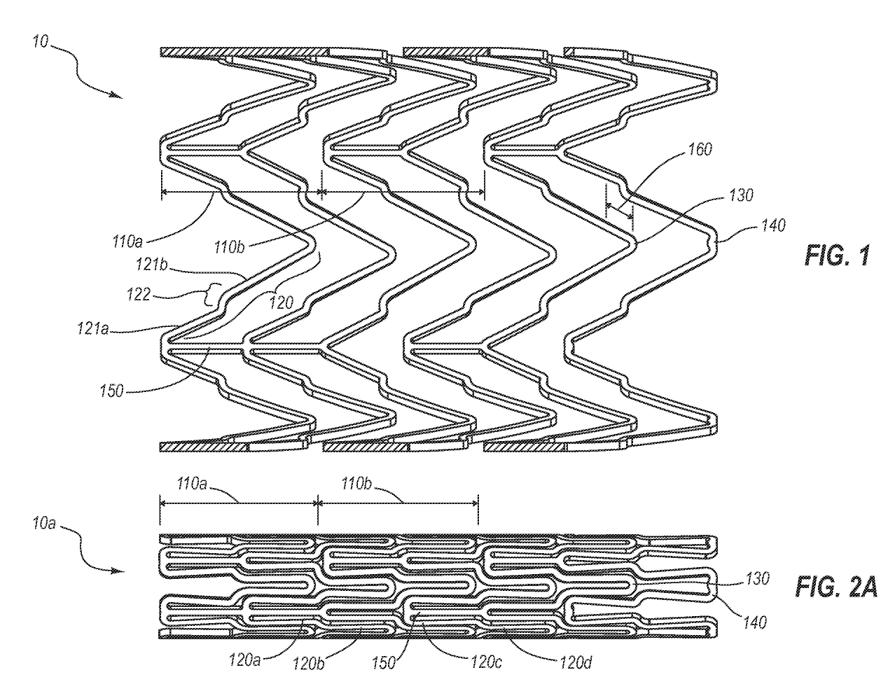

FIG. 1 is a side view of a flattened portion of an embodiment of a fatigue-resistant implantable medical device 10. The fatigue-resistant implantable medical device illustrated in FIGS. 1-3F is a stent, but it will be understood that the benefits and features of the present invention are also applicable to other types of implantable medical devices known to those skilled in the art.

For purposes of clarity and not limitation, the stent 10 is illustrated in a planar format. As shown, the stent 10 can include a plurality of annular elements 110 aligned longitudinally adjacent to each other along a longitudinal axis. Although the illustrated embodiment includes many interconnected annular elements, it is possible that an implantable medical device include one or a plurality of annular elements 110. As depicted in FIG. 1, at least a first annular element 110a and a second annular element 110b are identified.

Each annular element 110 can include a set of interconnected strut elements, shown as strut crossbars 120, which are disposed circumferentially about a longitudinal axis. In the depicted embodiment, each crossbar 120 includes first and second crossbar sections 121a and 121b and a bent section 122 that couples the first and second crossbar sections 121a and 121b. Accordingly, the implantable medical device 10 can include a plurality of annular elements 110 that can have a plurality of crossbars 120 that are connected together by elbows 130 having a first configuration and elbows having a second configuration 140. Adjacent annular elements (e.g., 110a and 110b) can be joined together by linkage elements 150 that join elbows 130 and 140 together. In the illustrated configuration, the stent 10 has a generally sinusoidal pattern that allows the stent 10 to expand in order to scaffold a body lumen. In addition, because the adjacent annular elements (e.g., 110a and 110b) are not connected by linkage elements 150 at every elbow 130 and 140, the design has a nesting distance shown schematically at 160. Nesting distance 160 allows adjacent annular elements (e.g., 110a and 110b) to nest together or stretch apart in the expanded configuration so as to shorten or lengthen the stent 10 without also changing the diameter of the stent. As such, the stent 10 can be shape-set at a length that is shorter or longer than its laser-cut length so as to allow the stent 10 to be deployed at a length that is greater than or less than its laser cut (i.e., unconstrained) length. As discussed above, a stent (e.g., stent 10) that is deployed to a length that is greater or less than its laser-cut length will experience reduced fatigue and will have a longer fatigue life when implanted in a vessel that is also lengthened or shortened prior to deployment.

It will be understood that annular elements 110 and linkage elements 150 can have other configurations while providing flexibility to the implantable medical device 10. That is, the sinusoidal design depicted in herein is merely an illustrative example. One of ordinary skill in the art will naturally appreciate that other stent designs can be used in conjunction with the present disclosure so long as the stent design can accommodate changes in length without changing in diameter.

Accordingly, one embodiment of the present invention includes a fatigue-resistant implantable medical device. The fatigue-resistant implantable medical device includes a tubular stent that includes a multiplicity of separate cuts that permit the tubular stent to expand to provide scaffolding support to a lumen. The tubular stent can have an unconstrained configuration that includes a first length and a constrained configuration that includes a second length that is greater than or less than the first length.

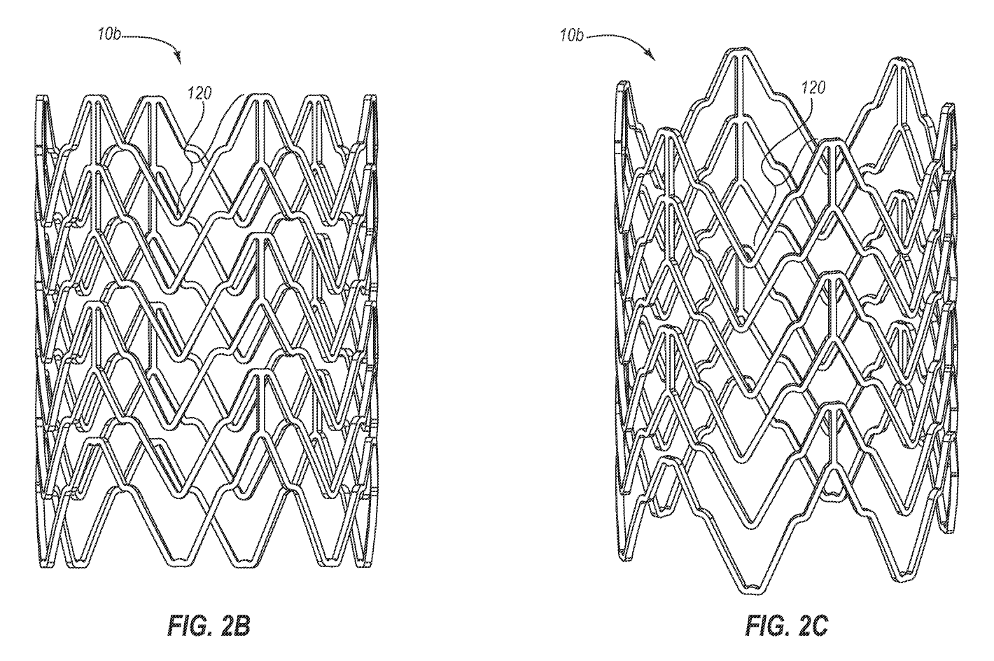

FIG. 2A shows a stent 10a in a collapsed configuration. Examples of stents in a collapsed configuration can include laser-cut stent that have not been expanded and heat set and stent that are constrained (i.e., collapsed) into a delivery configuration by a delivery sheath. When a stent such as stent 10a is in a collapsed configuration, strut crossbars 120 of are tightly packed and nearly parallel (see, e.g., 120a and 120b) along the circumference of stent 10a. Strut crossbars (see, e.g., 120a and 120b relative to 120c 120d) are also tightly packed and nearly parallel in adjacent annular rings 110a and 110b. Because of this tight packing, the length of stent 10a is essentially fixed in the collapsed configuration. As such, if one were to attempt to axially shorten stent 10a (i.e., compress it horizontally), strut crossbar 120a, for example, would butt up against strut crossbar 120c, for example, from adjacent rings 110a and 110b. As a result, the ability of stent 10a to change its length in the collapsed configuration is relatively limited.

FIGS. 2B and 2C show views of a stent 10b in an expanded configuration. As can be seen in FIGS. 2B and 2C, when stent 10b is expanded to a larger diameter, the pattern of crossbars 120 opens up and the pattern of crossbars is much less densely packed. This loosely packed configuration allows adjacent rings 110a and 110b to move axially. The net result is that, unlike the collapsed configuration seen in FIG. 2A, an expanded stent such as stent 10a can change its length by a percentage.

Moreover, as will be discussed in greater detail below, stent 10b can be shape-set in an expanded configuration at a length that is shorter or longer than the collapsed configuration (e.g., a laser-cut configuration) shown in FIG. 2A. When such a stent is placed into a collapsed configuration, such as into a delivery configuration, the pattern of strut crossbars (see, e.g., 120a-120d) can force the stent to elongate or shorten and return to its laser-cut length (i.e., pre shape-set length). In other words, compressing the stent typically causes the stent to return to is collapsed configuration, such as illustrated in FIG. 2A.

As such, in one embodiment of the present invention, the unconstrained configuration can include a shape-set and expanded state. The constrained configuration can include a delivery configuration wherein the tubular stent is radially compressed onto a delivery catheter by a delivery sheath. The constrained configuration can also include a deployed configuration wherein the tubular stent is deployed in a body lumen.

In one embodiment, the second length (i.e., the length in the constrained configuration) can be about 2% to about 15% different (i.e., longer or shorter) than the first length (i.e., the length in the unconstrained configuration). In another embodiment, the second length can be about 3% to about 10% different (i.e., longer or shorter) than the first length or, in another embodiment, the second length can be about 5% different (i.e., longer or shorter) than the first length.

The tubular stent may be formed from a shape-memory material (“SMM”) and/or a super-elastic material. For example, the SMM can be formed in a manner that allows for restriction to collapse or constrain the stent into a delivery configuration within a delivery catheter. But the stent can automatically expand once extended from the delivery catheter. As such, in one embodiment, the tubular stent can be a self-expanding stent. SMMs have a shape memory effect in which they can be made to remember a particular shape. Once a shape has been remembered, the SMM may be bent out of shape or deformed and then returned to its original shape by unloading from strain or heating. Typically, SMMs can be shape memory alloys (“SMA”) comprised of metal alloys, or shape memory plastics (“SMP”) comprised of polymers. The materials can also be referred to as being superelastic.

Usually, an SMA can have any non-characteristic initial shape that can then be configured into a memory shape by heating the SMA and configuring the SMA into the desired memory shape. After the SMA is cooled, the desired memory shape can be retained. This allows for the SMA to be bent, straightened, compacted, and placed into various contortions by the application of requisite forces; however, after the forces are released, the SMA can be capable of returning to the memory shape. The temperatures at which SMAs and similar alloys change their crystallographic structure are characteristic of the alloy, and can be tuned by varying the elemental ratios or by the conditions of manufacture.

Shape memory materials are characterized by their austenite and martensite states. The transformation between austenite and martensite is reversible but the temperature at which it occurs is different whether the shape memory alloy is being cooled or heated. This difference is referred to as the hysteresis cycle. This cycle is characterized by four different temperatures: Aₛ (Austenite Start), A_{f} (Austenite Finish), Mₛ (Martensite Start), and M_{f} (Martensite Finish). A martensitic shape memory alloy will begin to transform to austenite when its temperature reaches Aₛ and will be fully austenitic when the temperature reaches A_{f}. Upon cooling from a high temperature, martensite will start to appear when the temperature reaches Mₛ and the transformation will be complete when the temperature drops below M_{f}. A number of parameters including alloy composition and thermo-mechanical history can affect the transformation temperatures and can be adjusted for specific applications.

Suitable examples of shape-memory and/or super-elastic materials that can be used in the present invention include, but are not limited to, copper-zinc-aluminum; copper-aluminum-nickel; and nickel-titanium (“NiTi”) alloys known as nitinol. Cobalt-chromium-nickel alloys and cobalt-chromium-nickel-molybdenum alloys (known as elgiloy alloys) are similar to SMAs in that they have a high modulus of elasticity and they can be used in many similar applications. However, unlike SMAs, cobalt-chromium-nickel alloys and cobalt-chromium-nickel-molybdenum can be permanently deformed without the application of heat by exceeding the modulus of elasticity. In a preferred embodiment, the shape-memory and/or super-elastic material is a nickel-titanium alloy.

Shape memory materials possess unique characteristics that are particularly useful in applications involving implantable medical devices including endoprosthetic devices. If a piece of a shape memory alloy, such as nitinol, is mechanically stretched, compressed, bent, or twisted in its martensitic phase, it will return to its original configuration upon heating. Typically, the shape of the shape memory alloy is set to by deforming an austenitic material at high temperature, cooling the material to a martensitic state. When the material is again heated above the A_{f} temperature, the material will return to the shape it had when it was deformed in the austenitic state.

### III. Method of Manufacturing

In one embodiment, a method of manufacturing a fatigue-resistant implantable medical device is disclosed. In one embodiment, a method of manufacturing a fatigue-resistant implantable medical device can include a method for manufacturing a fatigue resistant, self-expanding stent. A typical procedure for manufacturing a self-expanding stent includes starting with a drawn tube formed from a material such as a Ni—Ti alloy, laser cutting the tube to form a tubular stent having a pattern that will allow the tubular stent to expand and flexibly scaffold a vessel, expanding and heat-setting the tubular stent to shape-set the stent in an expanded configuration, and compressing or “crimping” the expanded and heat-set tubular stent onto a delivery catheter.

Accordingly, the method of manufacturing a fatigue-resistant implantable medical device can include (1) providing a tubular stent configured to expand between a first configuration and a second configuration, the first configuration including a first diameter and a first length, (2) expanding the tubular stent to the second configuration, the second configuration including a second diameter that is larger than the first diameter and a second length that is shorter or longer than the first length, (3) heat setting the tubular stent in the second configuration, and (4) radially compressing the tubular stent to return the tubular stent to the first configuration.

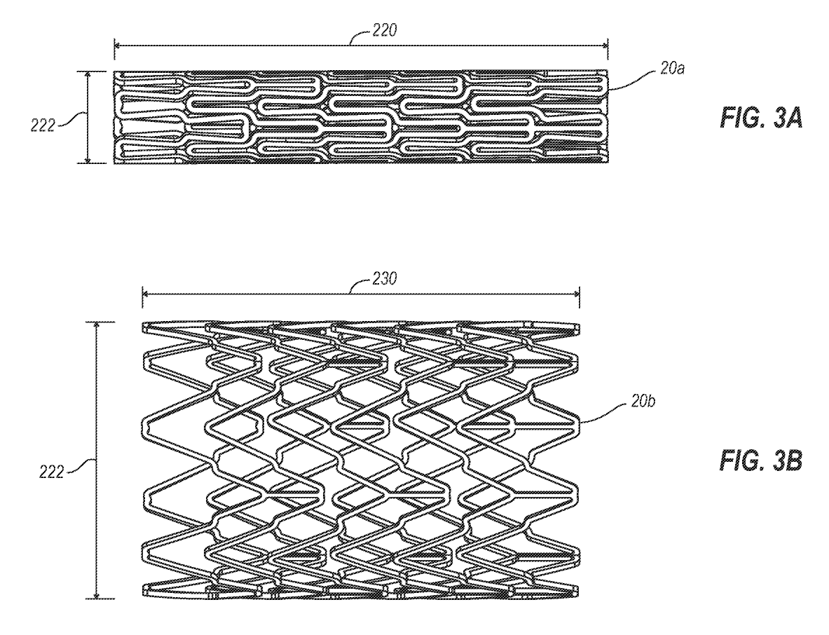

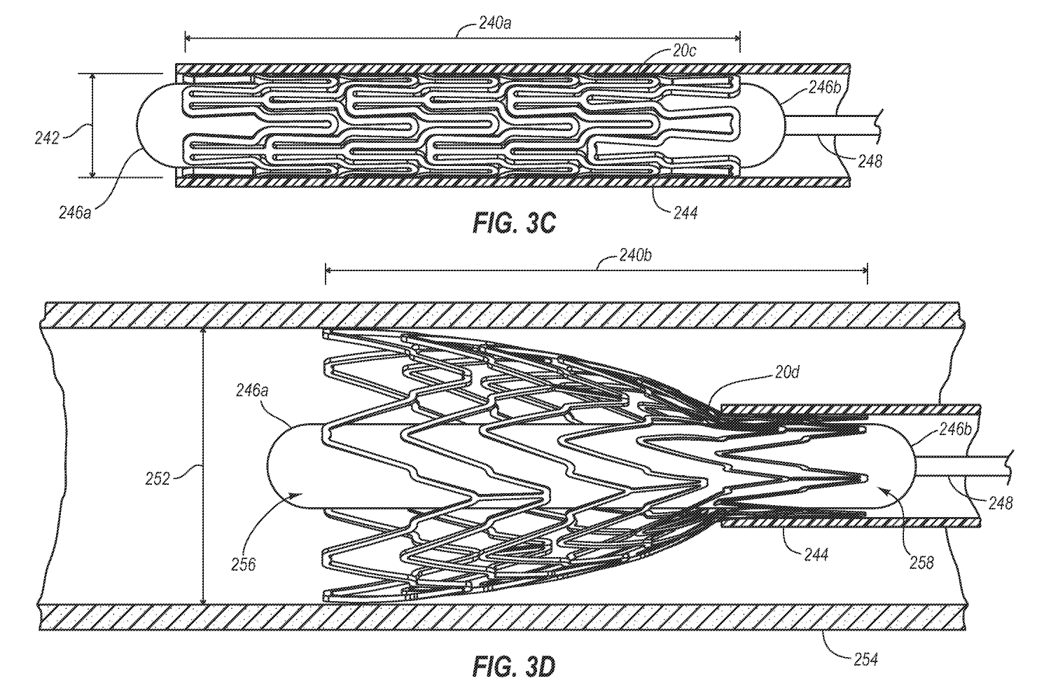

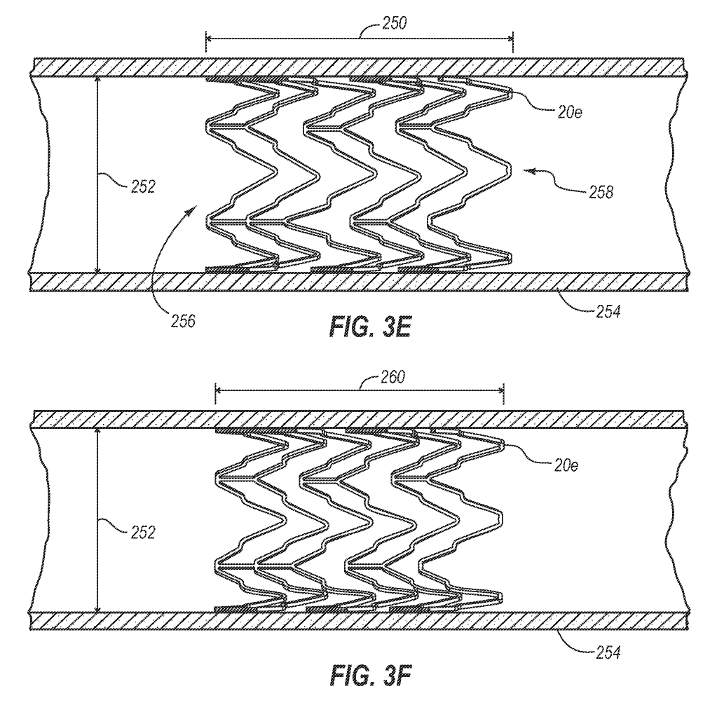

FIG. 3A illustrates a tubular stent having a first configuration. In one embodiment, a tubular stent having the configuration can include a laser-cut stent 20a. The laser-cut stent 20a has a length 220 and a diameter 222.

FIG. 3B illustrates stent 20b having a second configuration. After laser cutting, stent 20a can be expanded and shape-set (i.e., heat-set) at a diameter 232 that is greater than diameter 222. Because stent 20b is axially flexible (in contrast to stent 20a), stent 20b can also be shape-set to have a length 230 that is less than length 220. In one embodiment, the length of stent 20b can be shape-set to a length 230 that is about 2% to about 15% shorter than the length 220. In another embodiment, the length of stent 20b can be shape-set to a length 230 that is about 3% to about 10% shorter than the length 220 of stent 20a or, in another embodiment, the length of stent 20b can be shape-set to a length 230 that is about 5% shorter than the length 220. While length 230 is less than length 220 in the illustrated embodiment, one will appreciate that the stent 20b can also be shape-set at a length that is greater than the laser cut length for applications where it is desirable to implant a stent having a length shorter than its shape-set length (e.g., in a vessel that is shortened for stent deployment).

FIG. 3C illustrates a stent 20c in a constrained configuration. After expanding and shape-setting, stent 20b can collapsed to form a stent 20c in a delivery configuration. For instance, stent 20c can be crimped or collapsed onto a delivery catheter 246 with the help of a delivery sheath 244. Stent 20c has a diameter 242 that is small enough to allow stent 20c, the delivery catheter 246, an elongate guide catheter 248, and delivery sheath 244 to traverse the patient's vasculature in order to deliver stent 20c to a deployment site. In the embodiment shown in FIG. 3C, diameter 242 is substantially equal to diameter 222. One will appreciate however, that diameter 242 can be larger than diameter 222 provided that the diameter of stent 20c is small enough the traverse the patient's vasculature.

When stent 20c is placed into a constrained configuration, stent 20c elongates to length 240a. As stent 20c is constrained (e.g., compressed into a delivery configuration) stent crossbars (e.g., 120a-120d in FIG. 2A) and other features of the stent pattern butt up against one another, forcing stent 20c to return to a length that is equal to or substantially similar to its laser-cut length 220 (i.e., pre shape-set length).

### IV. Method for Deploying an Implantable Medical Device

Normally, stents and delivery systems are designed such that the laser-cut length, the shape-set length (i.e., the unconstrained length), the constrained/delivery configuration length, and the deployed length are essentially equal. Such stents deploy to their unconstrained length. However, it is often the case that vessels are lengthened or shortened in order to facilitate stent deployment. This creates a situation where the deployed stent is free of axial load only when the vessel is in the configuration that the vessel had when the stent was deployed (i.e., lengthened or shortened). In contrast, when the vessel is in a neutral position (i.e., somewhere between full elongation and a fully shortened state), the vessel has a length that is different than when the stent was deployed. This can place the stent under axial (i.e., compressive or stretching) loads that can contribute to stent failure.

For example, consider a situation in which a stent is deployed in the superficial femoral artery (“SFA”). A SFA may elongate by as much as about 10% or more when the leg is fully extended versus when it is at full flexion. Stents are typically deployed into the SFA while the leg is fully extended. Extending the leg typically facilitates stent deployment by, for example, elongating the vessel and reducing the tortuousness of the path that stent and the stent delivery catheter have to traverse in order to reach the deployment site. However, when the leg and thus the SFA is moved throughout its range of motion, the stent may change length, by way of example only, from 100% to 90% with a mean stent length of 95%. This yields an undesirable condition in which the stent experiences a peak strain of about 10% and a mean strain of about −5%.

Using the method for deploying a fatigue-resistant implantable medical device described herein can, for example, shift the peak-to-peak strain from about 0% to −10% to about +/−5% and shift the mean strain from about −5% to about 0%, thereby improving the fatigue life of the stent. While the total peak-to-peak strain of 10% cannot be reduced without restricting vessel movement, the strain region over which this alternating strain occurs can be shifted from about −5% to about 0% using the methods described herein. If this is done successfully the mean strain will be approximately 0%.

Accordingly, in one embodiment, a method of deploying a fatigue-resistant implantable medical device can include (1) providing a tubular stent configured to transition between a lengthened configuration and a shortened configuration, the tubular stent being shape-set in an elongated or a shortened configuration such that the shape-set configuration has substantially no lengthwise strain (2) providing a body lumen that can transition between an elongated state, a shortened state, and a mean state that is between the elongated state and the shortened state, and (3) deploying the tubular stent in the elongated or the shortened configuration in a body lumen with the body lumen in the elongated state or the shortened state such that the deployed stent has substantially no lengthwise strain when the body lumen is in the mean state.

In one embodiment, the tubular stent can be formed from a shape-memory and/or a super-elastic material. Suitable examples of shape-memory and/or super-elastic materials include, but are not limited to, nickel-titanium alloys. In one embodiment the tubular stent can be a self-expanding stent.

In one embodiment, the present invention further includes deploying the self-expanding stent in a patient's leg in a superficial femoral artery. Accordingly, deploying a stent in a superficial femoral artery according to the present invention can include (1) extending the patient's leg so as to elongate the superficial femoral artery, (2) inserting a delivery catheter into the patient's superficial femoral artery, the delivery catheter including the self-expanding stent compressed into a delivery configuration by a delivery sheath, the stent compressed into the delivery configuration being in the lengthened configuration, (3) positioning the delivery catheter at a site of occlusion in the superficial femoral artery or at the deployment site, and (4) withdrawing the delivery sheath and allowing the self-expanding stent to deploy in the superficial femoral artery in the lengthened configuration.

FIG. 3C illustrates a stent 20c constrained into a delivery configuration on a delivery catheter 246 with the help of a delivery sheath 244. As discussed above, when stent 20c is placed into a constrained configuration, stent 20c elongates to length 240a. As stent 20c is constrained (e.g., compressed into a delivery configuration) stent crossbars (e.g., 120a-120d in FIG. 2A) and other features of the stent pattern butt up against one another, forcing stent 20c to elongate. In one embodiment, constraining stent 20c can force it to return to its laser-cut length 220 (i.e., pre shape-set length).

Referring now to FIG. 3D, a partially deployed stent is illustrated. In the process of deployment, delivery sheath 244 is withdrawn allowing stent 20d to expand and deploy in the vessel. As the delivery sheath is withdrawn axially from the first end 256 to the second end 258 of stent 20d, the exposed portion of the stent 20d is able to expand and engage with the walls of the vessel 254 while the compressed portion of stent 20d remains tightly compressed against the delivery catheter 246 by the delivery sheath 244. When the rings of stent 20d are withdrawn from the delivery sheath 244 and expand to engage with the vessel 254, they are generally unable to relax to their unconstrained length before their length is constrained by the vessel 254. As the sheath 244 continues to retract, stent 20d progressively expands and engages with the vessel 254. As such, stent 20d is unable to return to its unconstrained length 230 and stent 20d is deployed in a lengthened configuration (e.g., length 240b or 250), which can improve the fatigue life of the stent.

In one embodiment, the tubular stent can be deployed in the body lumen at a length 250 that is about 2% to about 15% longer than the length 230 of the shortened (i.e., the unconstrained, heat-set) configuration. In another embodiment, the tubular stent is deployed in the body lumen at a length 250 that is about 3% to about 10% longer than the length 230 of the shortened configuration or, in another embodiment, the tubular stent is deployed in the body lumen at a length about 5% longer than the length 230 of the shortened configuration.

FIG. 3E illustrates stent 20e in a deployed configuration having a diameter 252 in a vessel 254. Deployed diameter 252 is typically smaller than the diameter of the stent in the unconstrained configuration 232. For instance, a stent having an unconstrained diameter of 8 mm may be used to stent a vessel having a diameter of 4 mm. Deploying a stent in a vessel that is smaller than the diameter of the stent in its fully expanded, unconstrained configuration serves to ensure that the stent can scaffold the vessel while being able to accommodate expansion and contraction of the vessel. This practice also helps to prevent the stent from returning to its unconstrained length in the time between when the delivery sheath is extracted and when the stent engages with the vessel.

As discussed above, stents deployed in some vessels such as the SFA are generally deployed while the vessel is in an elongated state. When the vessel returns to its relaxed state following the stenting procedure, the stent can generally shortens along with the vessel. FIG. 3F illustrates a stent 20f deployed in vessel 254 has been allowed returned to its relaxed length allowing stent 20f to relax to length 260. In the example illustrated in FIG. 3F, length 260 is essentially equal to unconstrained length 230. As a result, stent 20f may experience less strain when vessel 254 is in a relaxed state as compared to a stent that is not deployed in an elongated (i.e., strained) configuration, which may improve the fatigue life of the stent.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A fatigue-resistant implantable medical device, comprising:
a tubular stent that includes a multiplicity of separate cuts that permit the tubular stent to expand to provide scaffolding support to a lumen;
the tubular stent having an unconstrained configuration that includes a first length and a constrained configuration that includes a second length that is greater than or less than the first length.

2. The fatigue-resistant implantable medical device recited in claim 1, the unconstrained configuration including a shape-set and expanded state.

3. The fatigue-resistant implantable medical device recited in claim 1, the constrained configuration including a delivery configuration wherein the tubular stent is radially compressed onto a delivery catheter by a delivery sheath.

4. The fatigue-resistant implantable medical device recited in claim 1, the constrained configuration including a deployed configuration wherein the tubular stent is deployed in a body lumen.

5. The fatigue-resistant implantable medical device recited in claim 1, the second length being about 2% to about 15% different than the first length.

6. The fatigue-resistant implantable medical device recited in claim 1, the second length being about 3% to about 10% different than the first length.

7. The fatigue-resistant implantable medical device recited in claim 1, the second length being about 5% longer than the first length.

8. The fatigue-resistant implantable medical device recited in claim 1, the second length being about 5% shorter than the first length.

9. The fatigue-resistant implantable medical device recited in claim 1, the tubular stent comprising a shape-memory and/or a super-elastic material.

10. The fatigue-resistant stent recited in claim 9, the tubular stent comprising a nickel-titanium alloy.

11. The fatigue-resistant implantable medical device recited in claim 1, the tubular stent being a self-expanding stent.

12. A method of manufacturing a fatigue-resistant implantable medical device, comprising:
providing a tubular stent configured to expand between a first configuration and a second configuration, the first configuration including a first diameter and a first length;
expanding the tubular stent to the second configuration, the second configuration including a second diameter that is larger than the first diameter and a second length that is shorter or longer? than the first length;
shape setting the tubular stent using heat in the second configuration; and
radially compressing the tubular stent to return the tubular stent to the first configuration.

13. The method as recited in claim 12, the tubular stent further including a tubular member having a multiplicity of separate cuts in a wall of the tubular member that permit the tubular stent to expand to provide scaffolding support to a lumen.

14. The method as recited in claim 12, the radially compressed stent further including a delivery configuration wherein the tubular stent is radially compressed onto a delivery catheter by a delivery sheath.

15. The method as recited in claim 12, the second length being about 2% to about 15% different than the first length.

16. The method as recited in claim 12, the second length being about 5% shorter than the first length.

17. The method as recited in claim 12, the second length being about 5% longer than the first length.

18. The method as recited in claim 12, the tubular stent comprising a shape-memory and/or a super-elastic material.

19. The method as recited in claim 18, the tubular stent comprising a nickel-titanium alloy.

20. The method as recited in claim 12, the tubular stent being a self-expanding stent.

21. A method of deploying a fatigue-resistant implantable medical device, comprising:
providing a tubular stent configured to transition between a lengthened configuration and a shortened configuration, the tubular stent being shape-set in an elongated or a shortened configuration such that the shape-set configuration has substantially no lengthwise strain;
providing a body lumen that can transition between an elongated state, a shortened state, and a mean state that is between the elongated state and the shortened state; and
deploying the tubular stent in the elongated or the shortened configuration in a body lumen with the body lumen in the elongated state or the shortened state such that the deployed stent has substantially no lengthwise strain when the body lumen is in the mean state.

22. The method as recited in claim 21, the tubular stent comprising a shape-memory and/or a super-elastic material.

23. The method as recited in claim 22, the tubular stent comprising a nickel-titanium alloy.

24. The method as recited in claim 23, the tubular stent being a self-expanding stent.

25. The method as recited in claim 24, further comprising deploying the self-expanding stent in a patient's leg in a superficial femoral artery.

26. The method as recited in claim 25, the deploying further comprising:
extending the patient's leg so as to elongate the superficial femoral artery;
inserting a delivery catheter into the patient's superficial femoral artery, the delivery catheter including the self-expanding stent compressed into a delivery configuration by a delivery sheath, the stent compressed into he delivery configuration being in the lengthened configuration;
positioning the delivery catheter at a site of occlusion in the superficial femoral artery; and
withdrawing the delivery sheath and allowing the self-expanding stent to deploy in the superficial femoral artery in the lengthened configuration.

27. The method as recited in claim 21, the tubular stent being deployed in the body lumen at a length about 2% to about 15% longer than the shortened configuration.

28. The method as recited in claim 21, the tubular stent being deployed in the body lumen at a length about 3% to about 10% longer than the shortened configuration.

29. The method as recited in claim 21, the tubular stent being deployed in the body lumen at a length about 5% longer than the shortened configuration.
